(19) United States
(12) Patent Application Publication
(10) Pub. No.: US 2023/0329969 A1
(43) Pub. Date: Oct. 19, 2023

(54) AUXILIARY APPLICATOR FOR A BIOSENSOR

(71) Applicants: Shaoda ZHANG, Guangdong (CN); Xingyu Zheng, Guangdong (CN); Jinhai Xian, Guangdong (CN)

(72) Inventors: Shaoda ZHANG, Guangdong (CN); Xingyu Zheng, Guangdong (CN); Jinhai Xian, Guangdong (CN)

(21) Appl. No.: 17/972,594

(22) Filed: Oct. 25, 2022

(30) Foreign Application Priority Data
Apr. 14, 2022 (CN) 202210387880.7

(51) Int. Cl. A61H 39/08

(52) U.S. Cl. CPC A61H 39/08; A61L 2202/24

(57) ABSTRACT

The invention relates to an auxiliary applicator for a biosensor, including an applicator body and a drying box, where the applicator body takes out a probe component from the drying box, and the applicator body includes: a launcher for fixing the probe component; a launching cylinder, where the launching cylinder is provided with a first elastic component, and the first elastic component is connected with the launcher, and is used to eject the launcher in a triggered state; and a needle returning component, where the needle returning component is provided with a second elastic component, and the second elastic component is simultaneously connected with the launcher and the guide needle. The invention not only reduces implantation time of the probe needle, but also speeds up withdrawal of the guide needle, thereby greatly reducing the user's pain.

### BACKGROUND OF THE INVENTION

The invention relates to the field of biosensors, in particular to an auxiliary applicator for a biosensor.

The diagnosis and treatment of many diseases need to monitor some physiological parameters at any time, such as body temperature, heart rate, blood pressure, blood oxygen, blood glucose, etc. Some of these parameters can be directly measured by physical methods; for example, temperature and heart rate can be measured in vitro at any time. However, some parameters cannot be measured in vitro; for example, for blood glucose monitoring of diabetic patients, blood or interstitial fluid must be measured.

The traditional blood glucose detection usually uses test paper and a glucometer to measure blood glucose by measuring the current generated by the reaction between glucose in blood and glucose oxidase in the test paper. The common blood glucose monitoring usually adopts the unpleasant fingertip detection method. In addition to repeated acupuncture for blood collection which causes the patient to be unpleasant, this method also requires instant blood glucose at seven time points: fasting, after breakfast, before lunch, after lunch, before dinner, after dinner, and before going to bed, which is very inconvenient. The instant blood glucose is easily affected by many factors, such as exercise, diet, drugs and mood fluctuation. It can only reflect the blood glucose at several time points of the day, but not throughout the day. It is difficult to find asymptomatic hyperglycemia and hypoglycemia, sometimes leading to serious side effects. Therefore, users who use traditional methods to measure blood glucose usually know the condition of blood glucose rise or fall too late.

Therefore, a method that can provide continuous blood glucose monitoring is needed, which enables patients to know their blood glucose status at any time and take measures in time, so that the disease can be controlled most effectively, complications can be prevented, and a higher quality of life can be obtained.

In order to obtain real-time data, the method of measuring interstitial fluid is an available continuous monitoring means in reality. A sensor is implanted into the blood vessel as a device carried by patients daily, which will not introduce high-risk conditions such as infection or blood loss, and can effectively provide necessary data, so it has become an important direction of clinical monitoring development.

The Chinese invention patent with the authorization announcement No. CN103750818B and authorization announcement date of Mar. 30, 2016 disclosed a subcutaneously implantable biosensor, which was implanted into the subcutaneous tissue for detection. When the existing implantable biosensors are implanted into the skin, because the implantable biosensors are very small, many of them are wrapped by hard needles and sent into the subcutaneous tissue together, and then the hard needles are withdrawn to leave the probe needle of the implantable biosensors in the subcutaneous tissue.

When the existing products implant hard needles and implantable biosensors, the implants used are slow in the implantation speed and inaccurate in the implantation position. The time spent on implantation and withdrawal during implantation is long, which makes users feel tingling and increases their pain. In addition, the slow implantation speed also causes damage to the subcutaneous tissue, which adversely affects the detection effect of the implantable biosensors.

The above problems are worth solving.

### BRIEF SUMMARY OF THE INVENTION

In order to overcome the shortcomings of the prior art, the invention provides an auxiliary applicator for a biosensor.

The technical solutions of the invention is as follows:

an auxiliary applicator for a biosensor includes an applicator body and a drying box, where the applicator body is used to take out a probe component from the drying box, and the applicator body includes:

a launcher for fixing the probe component;

a launching cylinder, wherein the launching cylinder is provided with a first elastic component, and the first elastic component is connected with the launcher, and is used to eject the launcher in a triggered state, so as to penetrate a guide needle in the probe component into a human body; and

a needle returning component, wherein the needle returning component is provided with a second elastic component, and the second elastic component is simultaneously connected with the launcher and the guide needle;

when the first elastic component ejects the launcher, the second elastic component is released, and the needle returning component pulls back the guide needle that pierces the human body.

The invention according to the above solutions is characterized in that the launcher is installed in the launching cylinder through a launcher bracket, the first elastic component includes a number of torsion springs and a torsion spring bracket for fixing the torsion springs, and the launcher bracket is clamped with the torsion spring bracket, and squeezes the torsion springs.

By adopting the above technical solutions, the squeezed torsion springs exerts a force on the launcher bracket, and when the launcher bracket is separated from the torsion spring bracket, the torsion springs release their elastic force and eject the launcher bracket.

Further, a hollow space in a middle of the torsion spring bracket is annular, and the launcher bracket penetrates through a hollow opening of the torsion spring bracket.

Further, the torsion spring bracket is provided with at least three torsion spring grooves, both sides of the torsion spring grooves are provided with clamping openings facing upwards to clamp two ends of the torsion springs, middle parts of the torsion spring grooves are provided with notches, and torsion arms of the torsion springs extend into a center of the torsion spring bracket from the notches.

By adopting the above technical solutions, multiple torsion springs exert a same force on the launcher bracket, so as to ensure that the launcher is ejected along a central axis of the launching cylinder, so as to better penetrate the probe needle into a detection part.

Further, an inner wall surface of the torsion spring bracket is provided with a bump, the launcher bracket is provided with a clip that can buckle the bump, a top of the clip is provided with an inclined side push surface, and the side push surface is higher than an upper edge of the torsion spring bracket.

By adopting the above technical solutions, when the side push surface of the clip is pushed by force, the clip is separated from the bump, so that the launcher bracket is disconnected from the torsion spring bracket, and the torsion springs on the torsion spring bracket eject the launcher bracket.

Further, a bottom of the torsion spring bracket is provided with a support foot, the launcher is provided with a clamping groove, and the support foot passes through the launcher bracket and is connected with the clamping groove; a contact surface between the support foot and the clamping groove is an inclined surface.

By adopting the above technical solutions, the leg of the torsion spring bracket fix the launcher in a bottom groove of the launcher bracket, so that the launcher and the launcher bracket can be separated while the launcher bracket is separated from the torsion spring bracket.

The invention according to the above solutions is characterized in that the needle returning component further includes a needle returning cap, the second elastic component is a spring, the spring is arranged in the needle returning cap, and one end of the spring abuts against the needle returning cap, the other end abuts against the launcher bracket, and the needle returning cap is further connected with the guide needle.

By adopting the above technical solutions, before the first elastic component ejects the launcher, the needle returning cap and the launcher bracket extrude the spring, and the needle returning component obtains power to draw back the guide needle through the spring.

Further, the needle returning cap is provided with a jaw for connecting the guide needle, and the jaw is composed of four opposite hooks; an end of the guide needle is provided with a clamping connector, and the four hooks are connected with the clamping connector.

Further, the clamping connector is conical.

According to the above solutions, the invention is characterized in that the applicator body further includes a handle piece, and the handle piece is connected with the launching cylinder to trigger the launching cylinder to eject the launcher.

Further, an inner wall of the handle piece is provided with an unlocking sheet, a fixed cylinder of the launching cylinder is provided with an unlocking groove, the unlocking sheet is slidably connected with the unlocking groove, and when the unlocking sheet slides to an unlocking position of the unlocking groove, the handle piece can press down and trigger the launching cylinder.

By adopting the technical solutions, the handle piece can move along the direction of the launching cylinder only when the unlocking piece is in the unlocking position on the unlocking groove; when the unlocking piece is in other positions on the unlocking groove, the handle piece is not unlocked, so the launching cylinder cannot be triggered, which can prevent the launching cylinder from being triggered by mistake, and causing the hard guide needle to eject prematurely and injuring users by mistake.

Further, the unlocking groove is in a step shape, including a first step and a second step, and the unlocking sheet slides along the first step to the second step and then is inserted into a deep groove of the second step.

By adopting the above technical solutions, the second step is the unlocking position. The unlocking groove has a simple structural design, high stability and easy operation.

Further, a lower part of the handle piece is provided with a trigger cap, and a bottom of the trigger cap abuts against the top of the clip of the launcher bracket.

The invention according to the above solutions is characterized in that the applicator body further includes a lower cover body, the lower cover body is threaded with the handle piece, an opening of the handle piece is provided with an external thread ring, an opening of the lower cover body is provided with a disposable internal thread ring, the external thread ring is connected with the disposable internal thread ring, and the disposable internal thread ring is provided with a breaking ring.

The invention according to the above solutions is characterized in that a top of the handle piece is provided with a crystal sticker.

The invention according to the above solutions is characterized in that the drying box includes a bottom box and a sealing film, a bottom of the bottom box is provided with a fixed snap bracket for storing the probe component, and a cavity of the bottom box is provided with a drying agent.

Further, the fixed buckle support includes a fastener and a support frame, the fastener includes an upper fastener and a lower fastener clamped to each other, and the lower fastener is connected to the bottom box; the support frame is arranged in the bottom box, the support frame passes through the lower fastener and the upper fastener from bottom to top, and the probe component is placed on a top of the support frame.

Further, the upper fastener and the lower fastener are both provided with a through hole, the through hole is adapted to a contour of the support frame, and the support frame passes through the through hole.

Further, an edge of the through hole of the upper fastener is provided with a probe fixing foot, a probe base of the probe component is provided with a fixing groove, and the probe fixing foot is clamped with the fixing groove.

Further, a side wall of the lower fastener is provided with a number of first bayonets, the first bayonets include an upper bayonet and a lower bayonet, a side wall of the upper fastener is provided with a number of first snap feet, and the first snap feet are clamped with the lower bayonet after being separated from the upper bayonet.

By adopting the technical solutions, in an initial state, the first snap feet of the upper fastener is clamped with the upper bayonet of the lower fastener; after the upper fastener is pressed into the lower fastener, the first snap feet of the upper fastener are clamped with the lower bayonet of the lower fastener.

Further, the probe base is provided with a second snap foot, an inner wall of a recess of the launcher is provided with a second bayonet, and the second snap foot is clamped with the second bayonet.

By adopting the above technical solutions, when the applicator body is inserted into the drying box, the probe component is embedded into the recess of the launcher, and the second snap foot of the probe base is clamped to the second bayonet on the inner wall of the recess of the launcher, so that the probe component is fixed in the recess of the launcher.

The invention according to the above solutions is characterized in that the probe component includes the guide needle, the probe base and a sensor core piece, wherein the guide needle and the sensor core piece are fixed on the probe base; the guide needle is provided with a needle slot, and a detection needle of the sensor core piece is arranged in the needle slot.

Further, the sensor core piece is provided with three conductive contacts, an end of the guide needle far away from a needle head is provided with a clamping connector, and the conductive contacts and the clamping connector are exposed outwards.

By adopting the technical solutions, when the probe base is combined with the launcher, the conductive contacts are connected with three contacts on a circuit board of the launcher; the clamping connector of the guide needle is inserted with the jaw of the needle returning cap inside the applicator body.

The invention according to the above solutions has the beneficial effects of:

In this invention, by setting two sets of elastic components with strong deformation recovery, the first elastic component (torsion spring) is used to rapidly penetrate the guide needle into the human skin, thereby implanting a probe needle of a biosensor; and the second elastic component (spring) is used to rapidly withdraw the guide needle, which shows that the invention not only reduces implantation time of the probe needle, but also speeds up withdrawal of the guide needle, thereby greatly reducing the user's pain;

Further, when the handle piece is not unlocked, the handle piece cannot trigger the launching cylinder, which can prevent the launching cylinder from being triggered by mistake, and causing the hard guide needle to eject prematurely and injuring the user by mistake, and can also prevent the core component from being damaged by premature ejection, and resulting in waste.

### BRIEF DESCRIPTION OF THE DRAWINGS

FIG. 1 is a structural diagram of an applicator body of the present invention;

FIG. 2 is a structure breakdown of an applicator body;

FIG. 3 is a structure breakdown of a launching cylinder inside an applicator body;

FIG. 4 is an enlarged view of part A in FIG. 3;

FIG. 5 is a schematic diagram of a torsion spring bracket and a launcher bracket;

FIG. 6 is a schematic diagram of a bottom view of a torsion spring bracket;

FIG. 7 is a structural diagram of a launcher;

FIG. 8 is a structure breakdown of a launcher;

FIG. 9 is a schematic diagram of a movement process of handle piece unlocking;

FIG. 10 is a structural diagram of a drying box without tearing a sealing film;

FIG. 11 is a structural diagram of a drying box after tearing a sealing film;

FIG. 12 is a structure breakdown of a drying box;

FIG. 13 is an enlarged view of part B in FIG. 8;

FIG. 14 is a schematic diagram of an applicator body inserted into a drying box;

FIG. 15 is a schematic diagram of an applicator body removed from a drying box;

FIG. 16 is a decomposition diagram of an upper fastener and a lower fastener;

FIG. 17 is a schematic diagram of a combination of an upper fastener and a lower fastener in an initial state;

FIG. 18 is a schematic diagram of an upper fastener after being pressed down.

In the figures, 1. Applicator body; 11. Handle piece; 111. External thread ring; 112. Crystal sticker; 113. Unlocking sheet; 12. Lower cap body; 121. Disposable internal thread ring; 13. Fixed cylinder; 131. Unlocking groove; 14. Trigger cap; 15. Needle returning cap; 151. Jaw; 16. Spring; 17. Torsion spring bracket; 171. Torsion spring groove; 172. leg; 173. Bump; 18. Torsion spring; 19. Launcher bracket; 191. Clip; 192. Side push surface;
2. Launcher; 21. Upper housing; 22. Lower housing; 221. Recess; 222. Second bayonet; 223. Clamping groove; 23. Circuit board; 24. Skin friendly sticker;
3. Drying box; 31. Bottom box; 311. Support frame; 32. Sealing film; 33. Upper fastener; 331. Probe fixing foot; 332. First snap foot; 34. Lower fastener; 341. First bayonet; 3411. Upper bayonet; 3412. Lower bayonet;
4. Probe component; 41. Guide needle; 411. Clamping connector; 42. Probe base; 421. Fixing groove; 422. Second snap foot; 43. Conductive contact.

### DETAILED DESCRIPTION OF THE INVENTION

In order to better understand the purpose, technical solutions and technical effects of the invention, the invention will be further explained below with reference to the accompanying drawings and embodiments. Meanwhile, it is stated that the embodiments described below are only used to explain the present invention rather than limit the present invention.

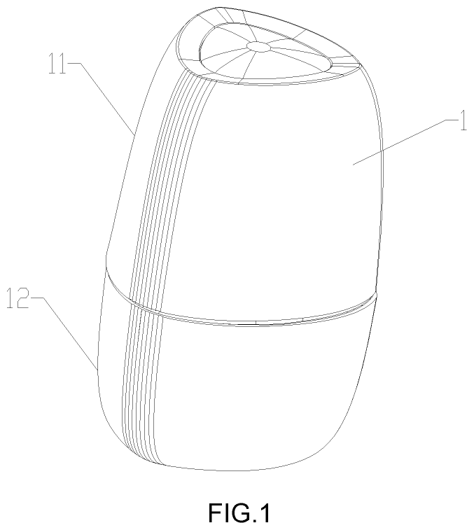

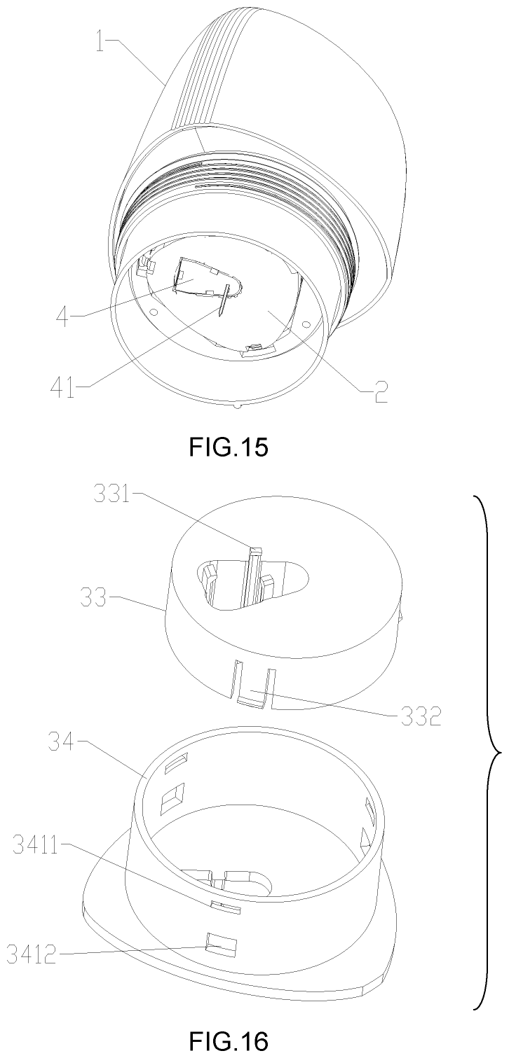

As shown in FIGS. 1 to 3, 14 and 15, an auxiliary applicator for a biosensor includes an applicator body 1 and a drying box 3, where the applicator body 1 is used to take out a probe component 4 from the drying box 3, the applicator body 1 includes a launcher 2, a launching cylinder and a needle returning component, the launcher 2 is used to fix the probe component 4, and the launching cylinder is used to eject the launcher 2 and penetrate the guide needle 41 in the probe component 4 into the human body; the needle returning component is connected with the guide needle 41, which is used to quickly withdraw the guide needle 41 after the guide needle 41 penetrates the human body.

In the invention, the launching cylinder is provided with a first elastic component, where the first elastic component is connected with the launcher 2 to eject the launcher 2 in a triggered state; the needle returning component is provided with a second elastic component, the second elastic component is simultaneously connected with the launcher 2 and the guide needle 41, and when the first elastic component ejects the launcher 2, the second elastic component is released, so that the needle returning component draws back the guide needle 41.

The first elastic component includes a torsion spring 18 and a torsion spring bracket 17, where the torsion spring 18 is fixed on the torsion spring bracket 17; the launcher 2 is installed in the launching cylinder through a launcher bracket 19; the launcher bracket 19 is clamped with the torsion spring bracket 17, and squeezes the torsion spring 18 on the torsion spring bracket 17, so that a torsion arm of the torsion spring 18 exerts a force on the launcher bracket 19; when the launcher bracket 19 is separated from the torsion spring bracket 17, the torsion spring 18 releases an elastic force and ejects the launcher bracket 19 and the launcher 2.

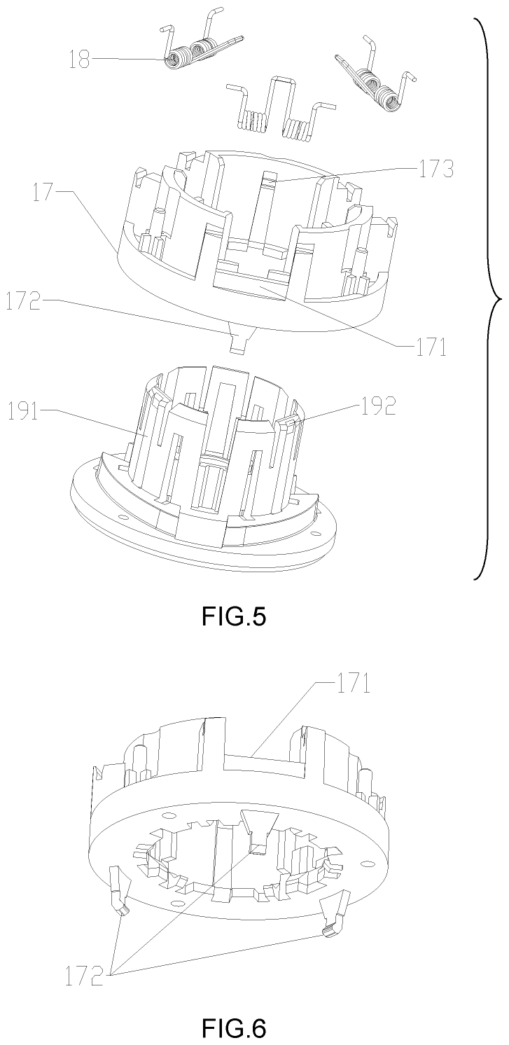

As shown in FIG. 5, a hollow space in a middle of the torsion spring bracket 17 is annular, and the launcher bracket 19 penetrates through the hollow space, a number of torsion springs 18 are uniformly installed on the torsion spring bracket 17, and torsion arms of the torsion springs 18 extend from the periphery to the middle. This structural design makes it easier to ensure that multiple torsion springs 18 exert a same force on the launcher bracket 19, so as to ensure that the launcher 2 is ejected along a central axis of the launching cylinder, so that the probe needle can be better punctured into a detection part.

The torsion spring bracket 17 is provided with at least three torsion spring grooves 171 for installing the torsion springs 18, and both sides of the torsion spring grooves 171 are provided with clamping openings facing upwards to clamp two ends of the torsion springs 18; a middle part of the torsion spring grooves 171 is provided with a notch for allowing a torsion arm to extend into a center of the torsion spring bracket 17. When the torsion spring bracket 17 is provided with three torsion spring grooves 171, the three torsion spring grooves 171 are distributed in a regular triangle.

In a preferred embodiment, an inner wall surface of the torsion spring bracket 17 is provided with a bump 173, while the launcher bracket 19 is provided with a clip 191 that can buckle the bump 173, a top of the clip 191 is provided with an inclined side push surface 192, and the side push surface 192 is higher than an upper edge of the torsion spring bracket 17 to facilitate contact with upper parts, thereby pushing the clip 191 away from the bump 173, so that the launcher bracket 19 is separated from the torsion spring bracket 17, and then the torsion spring can eject the launcher bracket 19 and launcher 2.

As shown in FIGS. 6 to 8, in a preferred embodiment, a bottom of the torsion spring bracket 17 is provided with a leg 172 for clamping the launcher 2, and correspondingly, the launcher 2 is provided with a clamping groove 223; during installation, after the launcher bracket 19 passes through the torsion spring bracket 17 from the middle, the leg 172 of the torsion spring bracket 17 passes through the launcher bracket 19 downward, and then clamps the launcher 2 to fix the launcher 2 in the bottom groove of the launcher bracket 19; since a contact surface between the leg 172 of the torsion spring bracket 17 and the clamping groove 223 of the launcher 2 is an inclined surface, the leg 172 only slightly blocks the launcher 2, and will not hinder the ejection of the launcher 2, and the torsion spring 18 ejecting the launcher bracket 19 can drive the launcher 2 to break off the leg 172 of the torsion spring bracket 17, and be ejected together with the launcher bracket 19; after ejection, the launcher 2 is not fixed by the leg 172 of the torsion spring bracket 17, and can be separated from the launcher bracket 19.

As shown in FIG. 5, the launcher bracket 19 is in a convex structure, with a bottom groove at the bottom for placing the launcher 2, a middle convex part for passing through the hollow opening of the torsion spring support 17, and its side wall for setting the clip 191. The size of the bottom groove is slightly larger than the size of the launcher, with its shape consistent with the launcher, and the bottom groove is only used to place the launcher, not fix the launcher; the outer periphery of the bottom groove is provided with a through hole, which passes through the leg 172 of the torsion spring bracket 17, and the leg 172 plays the role of fixing the launcher.

It can be seen that this embodiment has changed the traditional design idea. Instead of using the launcher bracket to fix the launcher, the leg of the torsion spring bracket are used to fix the launcher. When the launcher bracket is separated from the torsion spring bracket, the launcher and the launcher bracket are in a separable state, which makes it easy to take down the launcher bracket and only keep the launcher attached to the detection part of the human body.

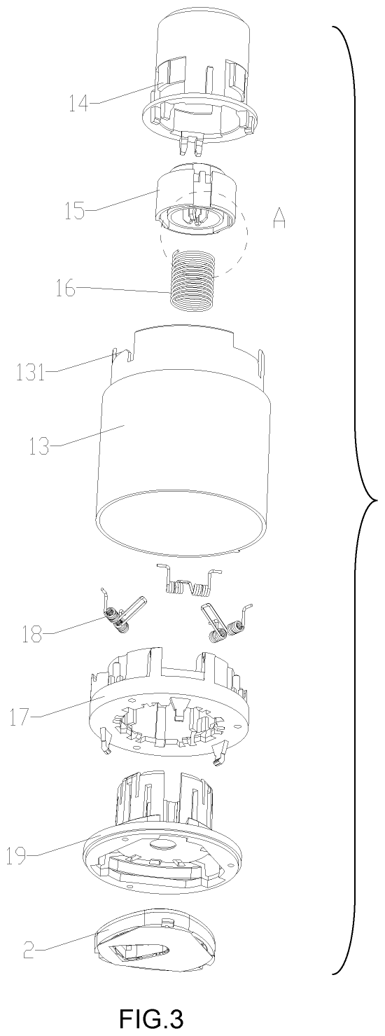

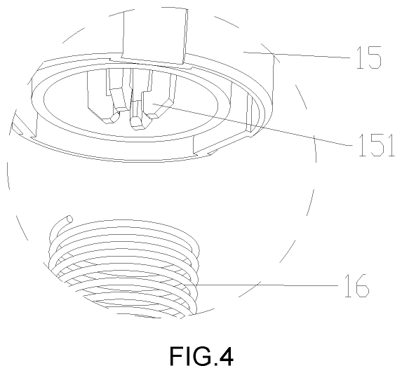

As shown in FIGS. 3 and 4, the needle returning component is provided with a second elastic component and a needle returning cap 15, which is connected with a guide needle 41; the second elastic component is a spring 16, which is arranged in the needle returning cap 15, and one end of the spring 16 abuts against the needle returning cap 15, and the other end abuts against the launcher bracket 19; before the first elastic component ejects the launcher 2, i.e., when the launcher bracket 19 is clamped with the torsion spring bracket 17, the needle returning cap 15 and the launcher bracket 19 extrudes the spring 16, and the extruded spring 16 has an elastic restoring force to provide a force for the withdrawal of the guide needle 41.

The needle returning cap 15 is provided with a jaw 151, which is composed of four opposite hooks with weak elasticity, and when the clamping connector 411 of the guide needle 41 is connected with the four hooks, the jaw 151 opens slightly until the clamping connector 411 is inserted and the hooks around it clamp the clamping connector 411; in order to better insert the guide needle 41 into the needle returning cap 15, the clamping connector 411 of the guide needle 41 is conical, and its tip end faces the needle returning cap 15. The size of the spring 16 is larger than the size of the four hooks of needle returning cap 15, and the four hooks are inserted into the spring 16.

To sum up, the applicator body is provided with two groups of elastic components, and the elastic release direction of the first elastic component faces the human body, which is used to eject the launcher to make the internal detection needle enter; the elastic force release direction of the second elastic component is far away from the human body, which is used to draw back the guide needle in a reverse direction, and the second elastic component is triggered instantaneously after the first elastic component is triggered. Therefore, the invention can quickly implant the probe needle of the biosensor into the human skin, and then quickly withdraw the guide needle from the skin, which not only reduces implantation time, but also speeds up withdrawal of the guide needle and reduces the user's pain.

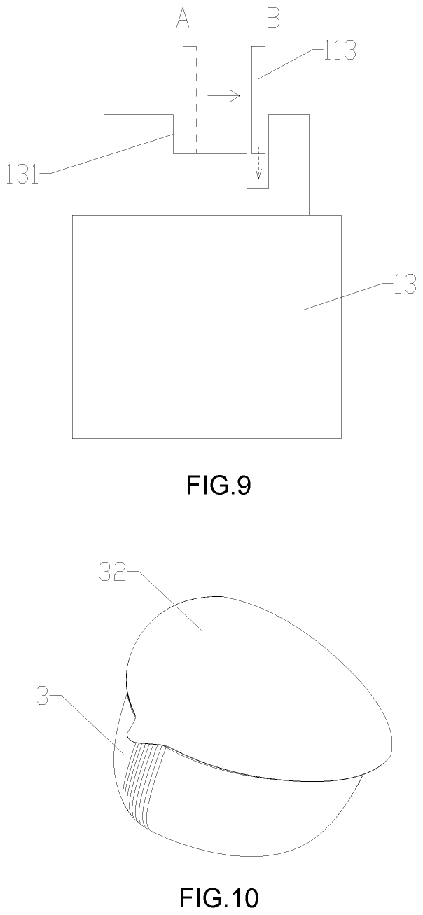

As shown in FIGS. 1, 2 and 9, the applicator body 1 further includes a handle piece 11, an inner wall of the handle piece 11 is provided with an unlocking sheet 113, the fixed cylinder 13 of the launcher cylinder is provided with an unlocking groove 131, the unlocking sheet 113 is slidably connected with the unlocking groove 131, and when the unlocking sheet 113 slides to an unlocking position of the unlocking groove 131, the unlocking is completed, and the handle piece 11 can only press down to trigger the launcher, while the handle piece 11 in a locked state cannot trigger the launcher cylinder, which can prevent the launcher cylinder from being triggered by mistake, and causing the hard guide needle 41 to eject prematurely and injuring the user by mistake, and can also prevent the core components from being damaged due to premature ejection, and resulting in waste.

The handle piece 11 is covered on the fixed cylinder 13 and can rotate along an axis of the fixed cylinder 13, so that the unlocking sheet 113 slides on the unlocking groove 131; the unlocking groove 131 is in a step shape, and when the unlocking sheet 113 of the handle piece 11 moves from the first step (point A) to the second step (point B) of the unlocking groove 131, the unlocking sheet 113 can be inserted downward into a deep groove of the second step, so that the handle piece 11 can be pressed down towards the fixed cylinder 13 to achieve an unlocked state of the handle piece 11.

In an optional embodiment, the unlocking sheet 113 is integrally formed with the handle piece 11, and the unlocking sheet 113 is a square sheet vertically arranged on the inner wall of the handle piece 11.

A lower part of the handle piece 11 is provided with a trigger cap 14. The bottom of the trigger cap 14 abuts against a top of the clip 191 of the launcher bracket 19. When the handle piece 11 is pressed down in the direction of the fixing cylinder 13, the trigger cap 14 pushes the side push surface 192 of the clip 191, making the clip 191 separate from the bump 173 of the torsion spring support 17, so that the launcher bracket 19 is liberated, and the launcher bracket 19 is ejected by the torsion spring.

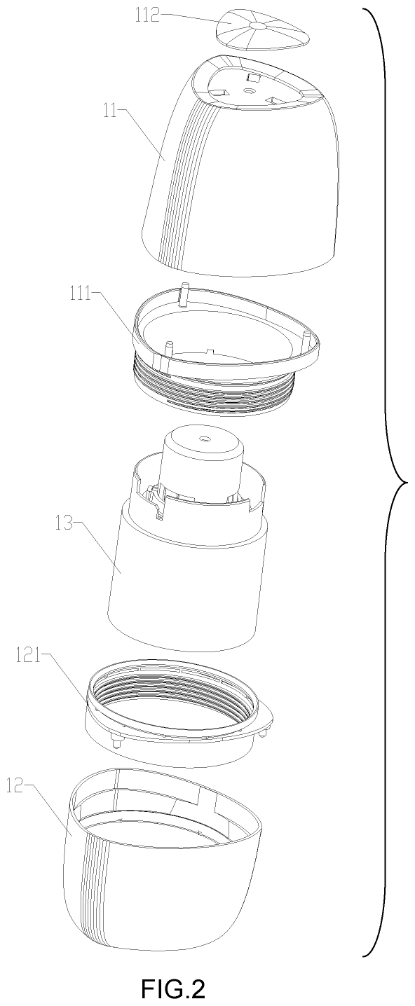

As shown in FIG. 2, in the present invention, the applicator body 1 further includes a lower cover body 12, which is threaded with the handle piece 11 to form a closed protective cavity to protect the launcher cylinder and core components of the applicator body 1. The opening of the handle piece 11 is provided with an external thread ring 111, and the opening of the lower cover body 12 is provided with a disposable internal thread ring 121. When the lower cover body 12 is unscrewed from the handle piece 11 for the first time, a breaking ring of the disposable internal thread ring 121 is disconnected and left at a ring groove of the external thread ring 111 to facilitate the user to judge that the applicator body 1 has been opened.

In an optional embodiment, the top of the handle piece 11 is provided with a crystal sticker 112 for displaying LOGO or decorative patterns.

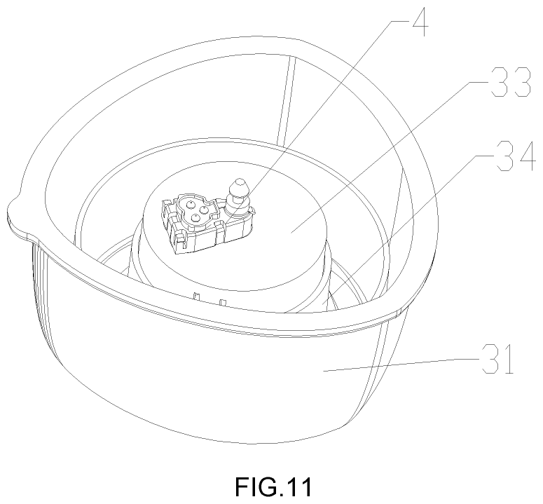

As shown in FIGS. 10 and 11, in the invention, the drying box 3 is used to store the probe component, including a bottom box 31 and a sealing film 32, and a bottom of the bottom box 31 is provided with a fixed snap bracket for storing the probe component; a drying agent is stored in the bottom box 31, which can dry the internal environment to prevent the probe component from being affected by moisture and affecting its performance.

As shown in FIGS. 11 to 16, in a preferred embodiment, in order to ensure that the probe component 4 can be smoothly transferred from the drying box 3 to the applicator body 1 after the applicator body is inserted into the drying box 3, the drying box 3 is designed with the following structure: the fixed snap bracket includes a fastener and a support frame 311, the fastener includes an upper fastener 33 and a lower fastener 34 clamped to each other, and the lower fastener 34 is connected to the bottom box 31; the support frame 311 is arranged at a center of the bottom box 31, both the lower fastener 34 and the upper fastener 33 are provided with a through hole, which is adapted to a contour of the support frame 311, and the support frame 311 passes through the lower fastener 34 and the upper fastener 33 from bottom to top; the probe component 4 is placed on the support frame 311, and the upper fastener 33 is exposed, and a probe base 42 of the probe component 4 is clamped by a probe fixing foot 331 of the upper fastener 33.

As shown in FIG. 16, a side wall of the lower fastener 34 is provided with a number of first bayonets 341, a side wall of the upper fastener 33 is provided with a number of first snap feet 332, and the first snap feet 332 are correspondingly connected with the first bayonets 341. In the solutions, the upper fastener 33 is inserted into an opening of the lower fastener 34, and the convex hooks of the first snap feet 332 are outwards connected with the first bayonets 341 of the lower fastener 34; in other alternative solutions, the upper fastener 33 can also be designed to cover the outside of the lower fastener 34, and at this time, the convex hooks of the first snap feet 332 are inwards connected with the first bayonets 341 of the lower fastener 34.

The first bayonets 341 include an upper bayonet 3411 and a lower bayonet 3412, and the upper bayonet 3411 is located above the lower bayonet 3412; the first snap feet 332 are a long strip with slight elasticity, and bottom surfaces of the convex hooks are provided with inclined surfaces, and the first snap feet 332 can be detached from the upper bayonet 3411 and then clipped into the lower bayonet 3412; the specific activity process is as follows: in an initial state, the first snap feet 332 of the upper fastener 33 are clamped with the upper bayonet 3411, and when the upper fastener 33 is pressed down, convex hook inclined surfaces of the first snap feet 332 is stressed, so that the first snap feet 332 are separated from the upper bayonet 3411; the upper fastener 33 is continuously pressed down until the first snap feet 332 are clamped into the lower bayonet 3412.

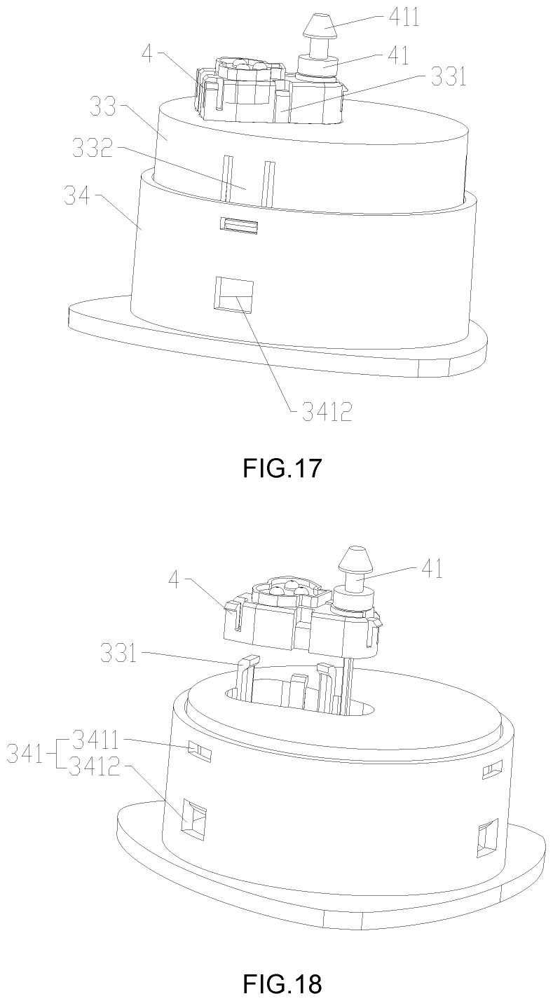

As shown in FIGS. 17 and 18, the probe fixing foot 331 is provided around the through hole of the upper fastener 33, the probe base 42 of the probe component 4 is provided with a corresponding fixing groove 421, and the fixing groove 421 is provided with an inclined surface. When the upper fastener 33 is pressed down, the probe fixing foot 331 moves downward, leaves the fixing groove 421 of probe base 42, and releases the probe component 4, and the probe component 4 can be taken away.

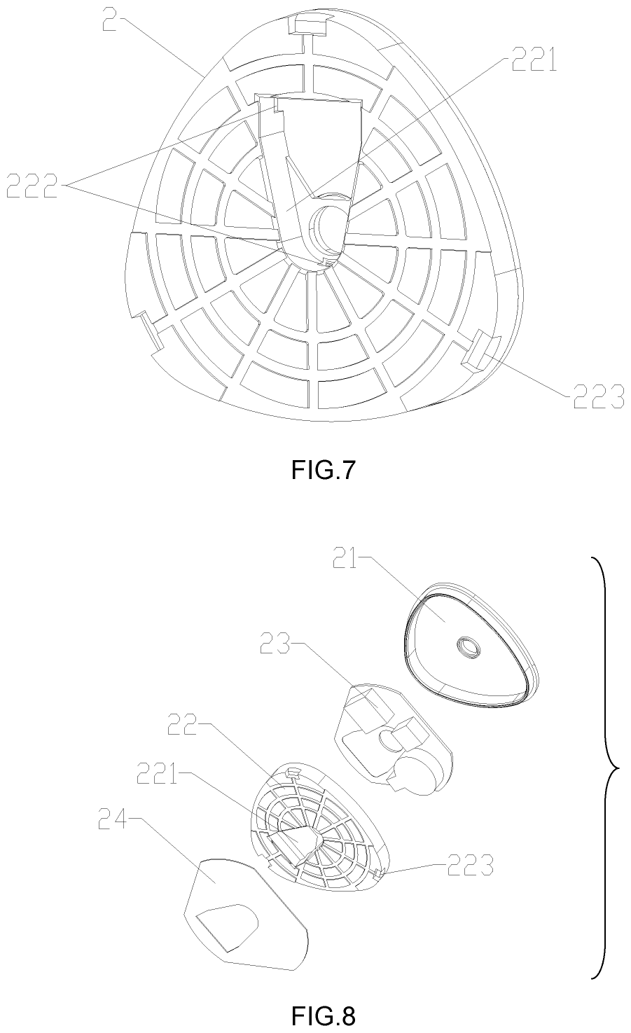

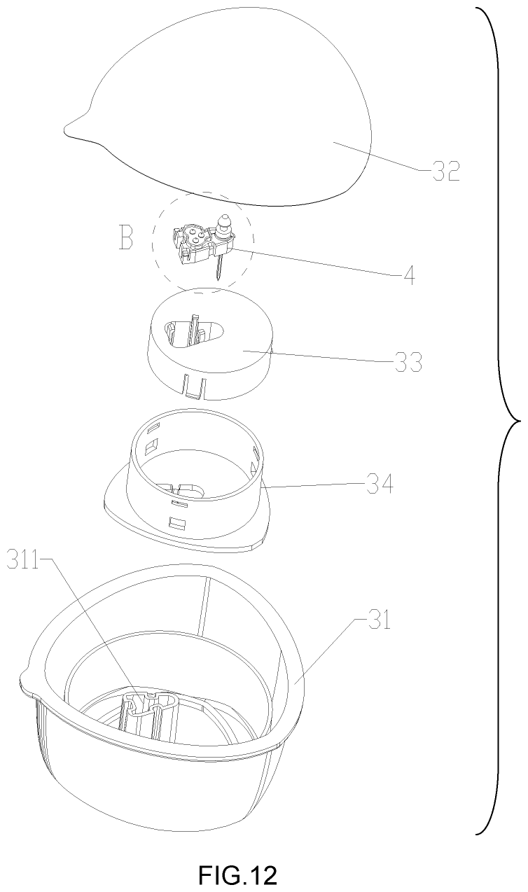

As shown in FIGS. 7 and 12, the probe base 42 is also provided with a second snap foot 422, and the inner wall of the recess 221 of the launcher is provided with a second bayonet 222 corresponding to the second snap foot 422, and when the applicator body 1 is inserted into the drying box 3, the probe component 4 is embedded into the recess 221 of the launcher, so that the second snap foot 422 is clamped with the second bayonet 222; after releasing the probe component 4 in the above way, the applicator body 1 is removed, the probe component 4 can be firmly fixed in the recess 221 of the launcher, and the probe component 4 is transferred from the drying box 3 to the applicator body 1.

In a preferred embodiment, both the applicator body 1 and the drying box 3 are triangular, which is convenient for users to grasp and apply force in use; and the triangular structure is more compact and convenient for storage.

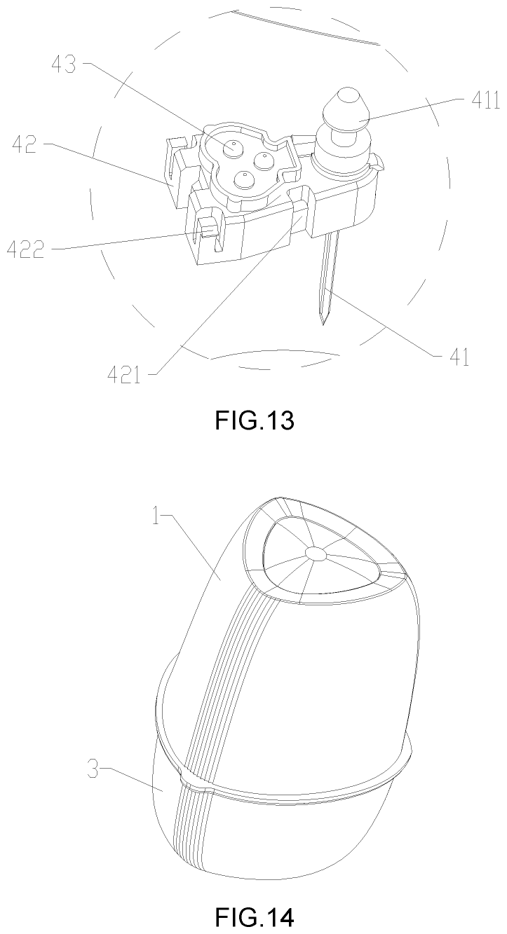

As shown in FIG. 13, in the invention, the probe component 4 includes a guide needle 41, a probe base 42 and a sensor core piece, where the guide needle 41 and the sensor core are fixed on the probe base 42 and combined with the launcher through the probe base 42; the sensor core piece is provided with three conductive contacts 43, and an end of the guide needle 41 far from a needle head is provided with a clamping connector 411; when the probe component 4 is stored in the drying box 3, the conductive contact 43 and the clamping connector 411 are exposed outwards, and when the probe base 42 is combined with the launcher, the conductive contact 43 can be connected with three contacts on a circuit board 23 of the launcher; the clamping connector 411 can be inserted and connected with the jaw 151 of the needle returning cap 15 inside the applicator body 1.

In the invention, the guide needle 41 is provided with a needle slot, and a probe needle of the sensor core piece is placed in the needle slot. When the guide needle 41 pierces the human skin, the probe needle can be sent into the skin, and after the guide needle 41 is pulled back, the detection part is left in the skin.

As shown in FIGS. 7 and 8, in the invention, the launcher 2 includes an upper housing 21, a lower housing 22 and a circuit board 23. The upper and lower housings protect the circuit board 23 in an inner cavity, and the lower housing 22 is provided with a recess 221, which is used to connect the probe component 4. According to the above solutions, an inner wall of the recess 221 is provided with the second bayonet 222, which is convenient for clamping with the fixing groove 421 of the probe base 42; the center of the launcher 2 is provided with a circular through hole, which is used to pass through the guide needle 41 and the detection needle of the sensor core piece. The launcher 2 is in the shape of a triangular sheet, and a surface of its lower housing 22 is pasted with a skin friendly sticker, which not only can be pasted on the human skin, but also has good comfort. It can be seen from the above solutions that when the launcher ejects the launcher bracket 19 and launcher 2, the launcher 2 and the launcher bracket 19 are in a disengaged state. At this time, the launcher is stuck to the human body through a skin friendly sticker 24, and the user can easily take away the launcher bracket 19.

It should be noted that when an element is referred as “fixed to” or “set to” another element, it can be directly on the other component or there can be a centered component. When an element is referred to as being “connected” to another element, it can be directly connected to another element or an intervening element may also be present.

The orientations or positions indicated by the terms “upper”, “lower”, “left”, “right”, “front”, “rear”, “vertical”, “horizontal”, “top”, “bottom”, “inside”, “outside” and the like are orientations or positions shown in the drawings, which are used only to facilitate description, and cannot be understood as a limitation to the technical solution.

The terms “first” and “second” are only used for descriptive purposes and cannot be understood as indicating or implying relative importance or implicitly indicating the number of technical features. “Multiple” means two or more, unless otherwise specifically defined.

The technical features of the above embodiments can be combined arbitrarily. For the sake of brevity, all possible combinations of the technical features in the above embodiments are not described. However, as long as there is no contradiction in the combination of these technical features, it should be considered as the scope of this specification.

The above embodiments only express some implementations of the present invention, and the descriptions thereof are relatively specific and detailed, but cannot be understood as limiting the scope of patent protection of the present invention. It should be noted that various modifications and improvements that can be made by those of ordinary skill in the art without departing from the concept of the present invention belong to the scope of protection of the present invention. Therefore, the scope of patent protection of the present invention shall be on the basis of the appended claims.

What is claimed is:

1. An auxiliary applicator for a biosensor, comprising an applicator body and a drying box, wherein the applicator body is used to take out a probe component from the drying box, and the applicator body comprises:
a launcher for fixing the probe component;
a launching cylinder, wherein the launching cylinder is provided with a first elastic component, and the first elastic component is connected with the launcher, and is used to eject the launcher in a triggered state, so as to penetrate a guide needle in the probe component into a human body; and
a needle returning component, wherein the needle returning component is provided with a second elastic component, and the second elastic component is simultaneously connected with the launcher and the guide needle;
when the first elastic component ejects the launcher, the second elastic component is released, and the needle returning component pulls back the guide needle that pierces the human body.

2. The auxiliary applicator for a biosensor according to claim 1, wherein the launcher is installed in the launching cylinder through a launcher bracket, the first elastic component comprises a number of torsion springs and a torsion spring bracket for fixing the torsion springs, and the launcher bracket is clamped with the torsion spring bracket, and squeezes the torsion springs.

3. The auxiliary applicator for a biosensor according to claim 2, wherein an inner wall surface of the torsion spring bracket is provided with a bump, the launcher bracket is provided with a clip that can buckle the bump, a top of the clip is provided with an inclined side push surface, and the side push surface is higher than an upper edge of the torsion spring bracket.

4. The auxiliary applicator for a biosensor according to claim 2, wherein a bottom of the torsion spring bracket is provided with a leg, the launcher is provided with a clamping groove, and the leg passes through the launcher bracket and is connected with the clamping groove; a contact surface between the leg and the clamping groove is an inclined surface.

5. The auxiliary applicator for a biosensor according to claim 1, wherein the needle returning component further comprises a needle returning cap, the second elastic component is a spring, the spring is arranged in the needle returning cap, and one end of the spring abuts against the needle returning cap, the other end abuts against the launcher bracket, and the needle returning cap is further connected with the guide needle.

6. The auxiliary applicator for a biosensor according to claim 5, wherein the needle returning cap is provided with a jaw for connecting the guide needle, and the jaw is composed of four opposite hooks; an end of the guide needle is provided with a clamping connector, and the four hooks are connected with the clamping connector.

7. The auxiliary applicator for a biosensor according to claim 1, wherein the applicator body further comprises a handle piece, and the handle piece is connected with the launching cylinder to trigger the launching cylinder to eject the launcher.

8. The auxiliary applicator for a biosensor according to claim 7, wherein an inner wall of the handle piece is provided with an unlocking sheet, a fixed cylinder of the launching cylinder is provided with an unlocking groove, and the unlocking sheet is slidably connected with the unlocking groove.

9. The auxiliary applicator for a biosensor according to claim 1, wherein the drying box comprises a bottom box and a sealing film, a bottom of the bottom box is provided with a fixed snap bracket for storing the probe component, the fixed snap bracket comprises a fastener and a support frame, the fastener comprises an upper fastener and a lower fastener clamped to each other, and the lower fastener is connected to the bottom box; the support frame is arranged in the bottom box, the support frame passes through the lower fastener and the upper fastener from bottom to top, and the probe component is placed on a top of the support frame.

10. The auxiliary applicator for a biosensor according to claim 9, wherein a side wall of the lower fastener is provided with a number of first bayonets, the first bayonets comprise an upper bayonet and a lower bayonet, a side wall of the upper fastener is provided with a number of first snap feet, and the first snap feet are clamped with the lower bayonet after being separated from the upper bayonet.
